(19) United States
(12) Patent Application Publication
(10) Pub. No.: US 2025/0367458 A1
(43) Pub. Date: Dec. 4, 2025

(54) LOAD MODULATION DETECTOR

(71) Applicant: Cochlear Limited, Macquarie University, NSW (AU)

(72) Inventor: Werner Meskens, Opwijk (BE)

(21) Appl. No.: 18/707,129

(22) PCT Filed: Dec. 1, 2022
(86) PCT No.: PCT/IB2022/061672

§ 371 Date: May 2, 2024

Related U.S. Application Data
(60) Provisional application No. 63/292,800, filed on Dec. 22, 2021.

(51) Int. Cl. A61N 1/372; A61N 1/02; A61N 1/36; A61N 1/378

(52) U.S. Cl. CPC A61N 1/3727; A61N 1/025; A61N 1/36038; A61N 1/37229; A61N 1/3787

(57) ABSTRACT

An apparatus includes load modulation sensing circuitry configured to detect variations on a DC electrical current used by power transmission circuitry configured to be in wireless communication with power receiving circuitry of a device. The load modulation sensing circuitry configured to detect at least positive variations on the DC electrical current greater than or equal to a first threshold level, to detect at least negative variations on the DC electrical current greater than or equal to a second threshold level, and to process detected positive variations and detected negative variations to generate signals indicative of load modulation of the power receiving circuitry of the device.

### BACKGROUND

### Field

The present application relates generally to systems and methods for facilitating wireless power transmission from a first device to a second device with wireless data transmission from the second device to the first device, and more specifically, for facilitating wireless power transmission from an external portion of a medical system to an implanted portion of the medical system and wireless data transmission from the implanted portion to the external portion.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises load modulation sensing circuitry configured to detect variations on a DC electrical current used by power transmission circuitry configured to be in wireless communication with power receiving circuitry of a device. The load modulation sensing circuitry configured to detect at least positive variations on the DC electrical current greater than or equal to a first threshold level, to detect at least negative variations on the DC electrical current greater than or equal to a second threshold level, and to process detected positive variations and detected negative variations to generate signals indicative of load modulation of the power receiving circuitry of the device.

In another aspect disclosed herein, a method comprises wirelessly transmitting power through tissue to an implant on or within a recipient's body by providing electrical current to power transmission circuitry inductively coupled to power reception circuitry of the implant. The method further comprises receiving a voltage indicative of variations imparted onto the electrical current by controlled adjustments of a resonant frequency and/or a resistive load of the power receiving circuitry. The method further comprises detecting the variations on the electrical current. Said detecting comprises generating, in response to the voltage, signals indicative of negative current variations and/or positive current variations on the electrical current and deriving data from the signals.

In another aspect disclosed herein, an apparatus comprises at least one coil driver comprising at least one current sense resistor. The apparatus further comprises at least one power transmitting coil configured to receive an electrical current from the at least one sense resistor, the at least one power transmitting coil configured to be in inductive communication with a device. The apparatus further comprises load modulation sensing circuitry configured to detect variations of a voltage across the at least one current sense resistor.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIGS. 1B-1D schematically illustrate other example cochlear implant auditory prostheses in accordance with certain implementations described herein;

FIG. 2A schematically illustrates an example apparatus in accordance with certain implementations described herein;

FIG. 2B schematically illustrates different load modulation events detectable for different SFTs in accordance with certain implementations described herein;

FIG. 3A schematically illustrates an example apparatus comprising load modulation sensing circuitry in accordance with certain implementations described herein;

FIG. 3B schematically an example apparatus in accordance with certain implementations described herein;

FIG. 3C schematically illustrates additional features of the example apparatus of FIG. 3B;

FIG. 3D schematically illustrates another example apparatus comprising load modulation sensing circuitry in accordance with certain implementations described herein;

FIG. 4A schematically illustrates an example first current sensing circuitry and first gain equation in accordance with certain implementations described herein;

FIG. 4B schematically illustrates an example second current sensing circuitry and a second gain equation in accordance with certain implementations described herein;

FIGS. 5A and 5B schematically illustrate an example first comparator circuitry and an example second comparator circuitry, respectively, in accordance with certain implementations described herein;

FIG. 6A schematically illustrates an example first current sensing circuitry comprising the at least one first amplifier of FIG. 4A and the first comparator circuitry of FIG. 5A in accordance with certain implementations described herein;

FIG. 6B schematically illustrates the second current sensing circuit comprising the at least one second amplifier of FIG. 4B and the second comparator circuitry of FIG. 5B in accordance with certain implementations described herein;

FIGS. 7A-7D schematically illustrate plots of the first voltage signals, second voltage signals, first digital signals and second digital signals from configurations configured to model various SFT values in accordance with certain implementations described herein; and

FIG. 8 is a flow diagram of an example method in accordance with certain implementations described herein.

### DETAILED DESCRIPTION

In certain implementations disclosed herein, a power transmitting apparatus comprises a demodulator configured to demodulate backlink signals of an inductive link (e.g., modulated RF signals on the electrical current provided to a power-transmitting RF coil antenna closely coupled to a power-receiving RF coil antenna in the near field, the modulated RF signals generated by load modulation of the power-receiving RF coil antenna). For implantable systems having an inductive (e.g., closely coupled) link between a power-transmitting external device (e.g., sound processor of a cochlear implant system) and a power-receiving implanted device (e.g., implant of the cochlear implant system), such backlink signals are affected by the thickness of the recipient's tissue (e.g., skin flap thickness) between the external and implanted devices. The demodulator utilizes separate positive and negative current variation detector circuits to detect positive and negative current changes and positive and negative transients for more reliable backlink signal detection for all recipients and skin flap thicknesses, as compared to conventional pulsed backlink telemetry.

The teachings detailed herein are applicable, in at least some implementations, to any type of implantable medical device (e.g., implantable sensory prostheses) comprising a first portion (e.g., external to a recipient) and a second portion (e.g., implanted on or within the recipient), the first portion configured to wirelessly transmit power to the second portion. For example, the implantable medical device can comprise an auditory prosthesis system utilizing an external sound processor configured to transcutaneously provide power to an implanted assembly (e.g., comprising an actuator). In certain such examples, the external sound processor is further configured to transcutaneously provide information (e.g., data signals; control signals) to the implanted assembly, which responds to the data by generating stimulation signals that are perceived by the recipient as sounds. In addition, the external sound processor can be configured to transcutaneously receive information (e.g., data signals; control signals) from the implanted assembly. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; etc. The concepts described herein and/or variations thereof can be applied to any of a variety of implantable medical devices comprising an implanted component configured to use magnetic induction to receive power (e.g., transcutaneously) from an external component and to store at least a portion of the power in at least one power storage device (e.g., battery; tank capacitor). The implanted component can also be configured to receive control signals from the external component (e.g., transcutaneously) and/or to transmit sensor signals to the external component (e.g., transcutaneously) while receiving power from the external component. In still other implementations, the teachings detailed herein and/or variations thereof can be utilized in other types of systems beyond medical devices utilizing magnetic induction for wireless power transfer. For example, such other systems can include one or more of the following: consumer products (e.g., smartphones; “internet-of-things” or IoT devices) and electric vehicles (e.g., automobiles).

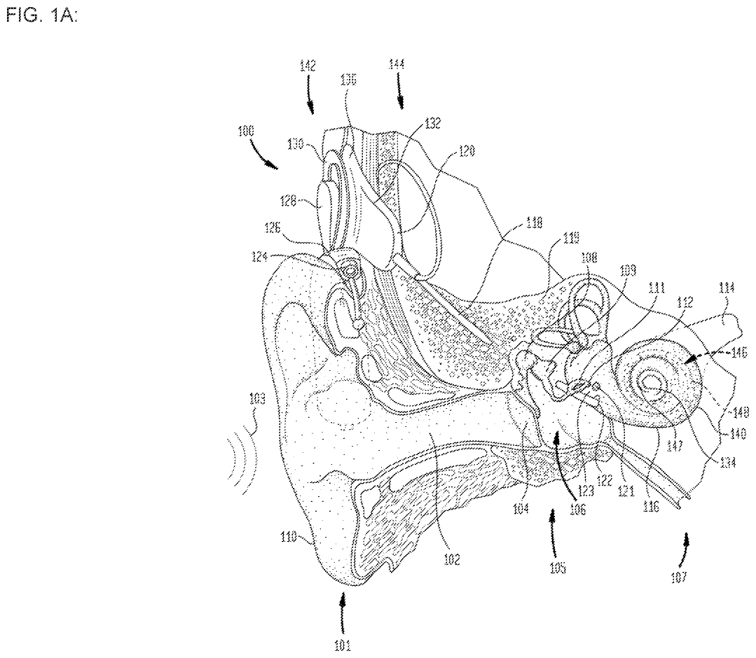

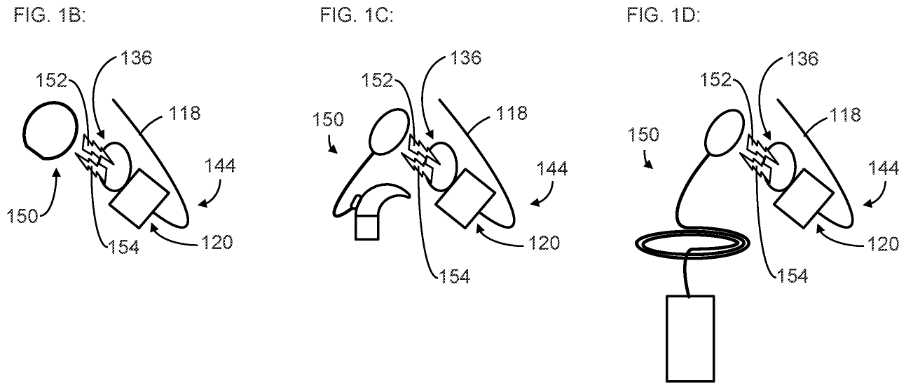

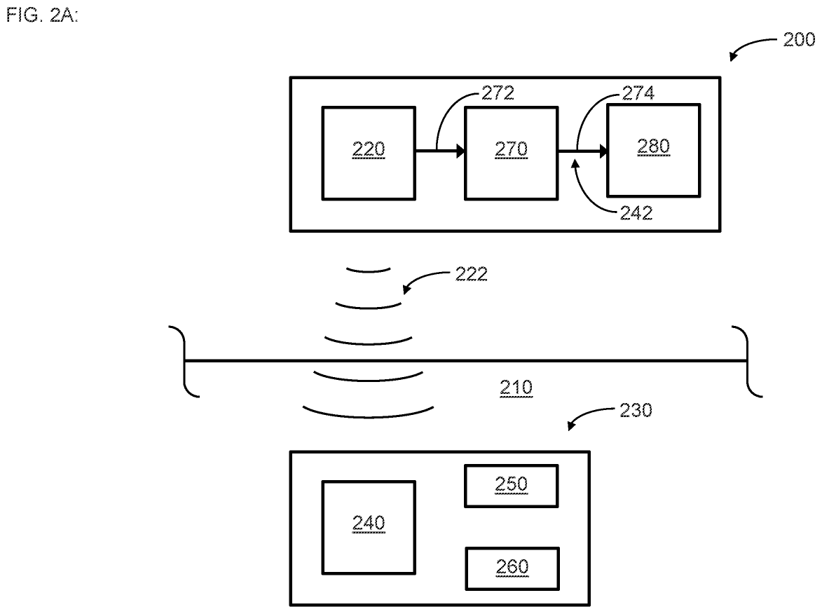

FIGS. 1A-1D show various example systems 100 compatible with certain implementations described herein. FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1A with a subcutaneously implantable assembly comprising an acoustic transducer (e.g., microphone), as described more fully herein.

As shown in FIG. 1A, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1A) (e.g., audio ports, such as a Direct Audio Input (DAI); data ports, such as a Universal Serial Bus (USB) port; cable ports, etc.). In the example of FIG. 1A, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1A) (e.g., battery; capacitor tank), a processing module (not shown in FIG. 1A) (e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1A, the external transmitter unit 128 comprises circuitry that includes at least one external inductive coil 130 (e.g., a wire antenna coil comprising multiple turns of electrically insulated copper wire). The external transmitter unit 128 also generally comprises a magnet (not shown in FIG. 1A) secured directly or indirectly to the at least one external inductive coil 130. The at least one external inductive coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1A, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive coil 136 (e.g., a wire antenna coil comprising multiple turns of electrically insulated single-strand or multi-strand platinum or gold wire), and generally, a magnet (not shown in FIG. 1A) fixed relative to the at least one internal inductive coil 136. The at least one internal inductive coil 136 receives power and/or data signals from the at least one external inductive coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIGS. 1B-1D schematically illustrate other example cochlear implant auditory prostheses 100 in accordance with certain implementations described herein. The example auditory prostheses 100 of FIGS. 1B-1D comprise an internal component 144 comprising an internal inductive coil 136, an internal microphone (not shown), and a stimulation unit 120 comprising an elongate stimulation assembly 118. The example auditory prostheses 100 of FIGS. 1B-1D further comprise an external device 150 configured to wirelessly transfer power 152 to the internal component 144 (e.g., to charge the internal component 144) and to receive load modulation backlink data 154 from the internal component 144 during the wireless power transfer. In FIG. 1B, the external device 150 comprises an “off-the-ear” (OTE) sound processing and charging device comprising sound processing circuitry and power transmitting circuitry (e.g., coil) within the same housing. In FIG. 1C, the external device 150 comprises a “behind-the-ear” (BTE) sound processing and charging device comprising sound processing circuitry and power transmitting circuitry (e.g., coil) within separate housings having a wired connection therebetween. Certain implementations of the auditory prosthesis 100 of FIGS. 1A-1C utilize load modulation to provide a low to medium data rate backlink (e.g., 41 bits/100 ms: 100 to 1000 bits per second) of certain information, while stimulation data from an external microphone 124 is transferred to the internal component 144 by other means (e.g., 2.4 GHz magnetic induction data transfer) or an implantable microphone (e.g., for TICI or MICI).

In FIG. 1D, the external device 150 comprises a dedicated charging device (e.g., the external device 150 does not provide stimulation data to the internal component 144). The external device 150 comprises power transmitting circuitry (e.g., coil) and a power source (e.g., configured to be worn on the recipient's body) within separate housings having a wired connection therebetween. In certain other implementations, the external device 150 comprises a wireless power transmission (WPT) charger (e.g., pillow charger) configured to wirelessly transfer power to the internal component 144 upon the recipient laying the body portion containing the internal component 144 on or within an operative region of the WPT charger.

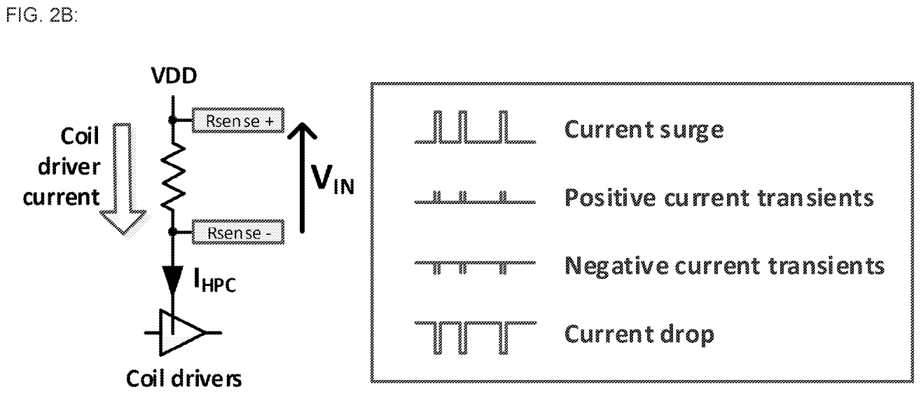

FIG. 2A schematically illustrates an example apparatus 200 in accordance with certain implementations described herein. The apparatus 200 (e.g., external device 150 of FIGS. 1A-1D) is configured to be external to a recipient's body 210 and comprises power transmission circuitry 220 in wireless communication with an implanted device 230 on or within the recipient's body 210 (e.g., the power transmission circuitry 220 can be configured to be inductively coupled to power receiving circuitry 240 of the implanted device 230 to transmit power 222). For example, the power transmission circuitry 220 can comprise at least one external coil (e.g., at least one external inductive coil 130) and at least one coil driver configured to provide a DC current to the at least one external coil, the at least one external coil can be inductively coupled to at least one internal coil of the power receiving circuitry 240 (e.g., at least one internal inductive coil 136). In certain implementations, the power receiving circuitry 240 can comprise at least one power storage element (e.g., at least one battery; at least one tank capacitor) configured to store the power wirelessly received from the apparatus 200 for subsequent use by the implanted device 230. The implanted device 230 can further comprise transducer circuitry 250 configured to provide stimulation signals to a portion of the recipient's body 210 and/or sensor circuitry 260 configured to generate sensor signals indicative of a detected characteristic of the recipient's body and/or the implanted device 230.

In certain implementations, the apparatus 200 is further configured to utilize load modulation for a data backlink from the implanted device 230 to the apparatus 200 during continuous wave power transfer (e.g., at 5 MHz or 6.78 MHz) from the apparatus 200 to the implanted device 230. Such load modulation can transfer data (e.g., at least 100 symbols per second) back from a power-receiving device (e.g., medical implant; transponder; tag) to a power-transmitting device (e.g., external portion of the medical implant; reader; interrogator) without the power-receiving device having an active transmitter. For example, the power receiving circuitry 240 can be configured to modulate a resonant frequency of the power receiving circuitry 240 (e.g., frequency detuning by about ±5% relative to the carrier frequency; switching or toggling between at least two resonant frequency values, such as about 5 MHz and about 7 MHz). For another example, the power receiving circuitry 240 can be configured to modulate a resistive load of the power receiving circuitry 240 (e.g., changing a Q factor of the inductive coupling; switching or toggling between two resistive load values). Such modulations of the power receiving circuitry 240 apply corresponding modulations to the transmitted power 222 and to the DC driver current inputted to the power transmission circuitry 220. These modulations can reflect (e.g., encode) information 242 (e.g., data signals; control signals) onto the DC driver current of the power transmission circuitry 220, effectively transmitting the information 242 from the implanted device 230 to the apparatus 200. The apparatus 200 further comprises load modulation sensing circuitry 270 configured to receive at least one signal 272 indicative of the modulations applied to the DC driver current and to generate digital signals 274 comprising the information 242.

As shown in FIG. 2A, the apparatus 200 can further comprise controller circuitry 280 configured to receive the digital signals 274 indicative of the load modulation, to extract (e.g., demodulate; decode) the information 242 from the digital signals 274, and to use the information 242 from the load modulation sensing circuitry 270. For example, the controller circuitry 280 can be configured to use the information 242 to identify aspects of the implanted device 230 (e.g., the type of implanted device 230; the identity of the implanted device 230; the battery type), to evaluate, report, and/or respond to an operational state of the implanted device 230 (e.g., implant voltage; implant current; battery state; battery voltage and charging/discharging current; battery charging level), to receive, evaluate, report, and/or respond to data from a sensor of the implanted device 230 (e.g., battery temperature; implant temperature; physiological data), to control operation of the apparatus 20 (e.g., restart link requests; synchronization acknowledgement; power control data), etc. The controller circuitry 280 can comprise one or more digital signal processors (DSPs), one or more microcontroller cores, one or more application-specific integrated circuits (ASICs), firmware, software, etc.

In certain implementations, the power transmission circuitry 220 and the power receiving circuitry 240 are stagger tuned (e.g., to achieve a predetermined data transfer bandwidth). For example, the transmitted RF power can have an average operational signal frequency of about 5 MHz, the power transmitting circuitry 220 can be tuned to a resonance frequency about 5% less than the average operational signal frequency (e.g., to about 4.75 MHz), and the power receiving circuitry 240 can be tuned to a resonance frequency about 5% more than the average operational signal frequency (e.g., to about 5.25 MHz).

Skin flap thickness (SFT) of the recipient's tissue between the apparatus 200 and the implanted device 230 varies among different recipients (e.g., in a range of 1 millimeter to 15 millimeters). The SFT can affect the modulations to the DC driver current inputted to the power transmission circuitry 220 (e.g., due to the impact of the coupling factor to the voltage/current transfer characteristics of the stagger tuned power transmission circuitry 220 and the power receiving circuitry 240). FIG. 2B schematically illustrates different load modulation events detectable for different SFTs in accordance with certain implementations described herein. The load modulation events can be detected by monitoring (e.g., sampling) a sense voltage across a current sense resistor in series with the power source and the power transmitting coils. The load modulation events have different characteristics depending on the SFT. For example, for small SFT values, detuning the at least one internal coil of the power receiving circuitry 240 (e.g., without modifying the resistive load) produces increases (e.g., surges) of the DC driver current provided to the at least one external coil of the power transmitting circuitry 220, while for medium or large SFT values, detuning the at least one internal coil of the power receiving circuitry 240 (e.g., without modifying the resistive load) produces reductions (e.g., drops) of the DC driver current provided to the at least one external coil of the power transmitting circuitry 220. In addition, for intermediate SFT values between the small SFT values and the medium or large SFT values, there are little or no net changes of the DC driver current by detuning the at least one internal coil of the power receiving circuitry 240 (e.g., without modifying the resistive load), but there are detectable positive and/or negative transients during the transitions of such detuning. If the SFT changes between large SFT values and small SFT values, the characteristics of the modulations on the DC driver circuit can change (e.g., surges become drops and vice versa). Furthermore, changes of the distance between the at least one external coil of the power transmission circuitry 220 and the at least one internal coil of the power receiving circuitry 240 (e.g., due to movements of the apparatus 200 relative to the implanted device 230) can produce variations (e.g., surges; drops) in the DC driver current, and these variations can be erroneously detected as part of the load modulation signals from the implanted device 230.

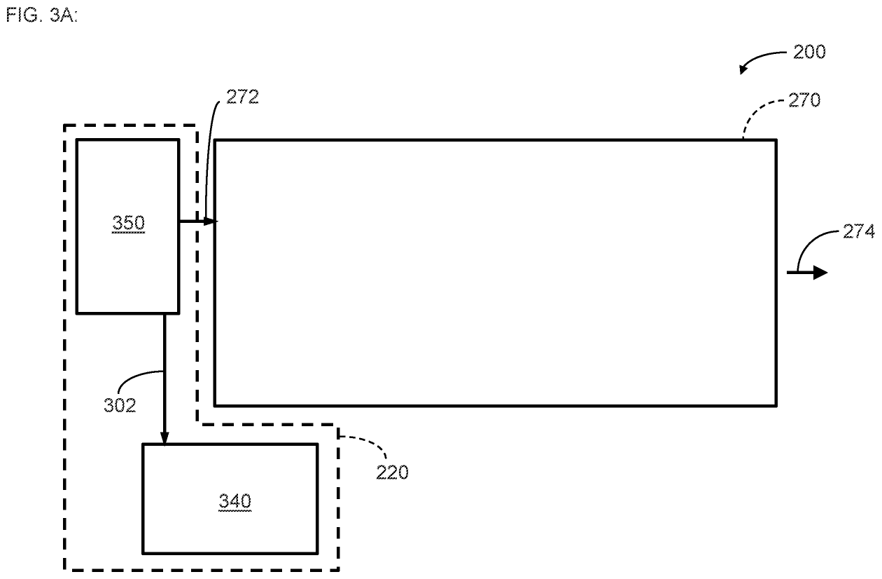

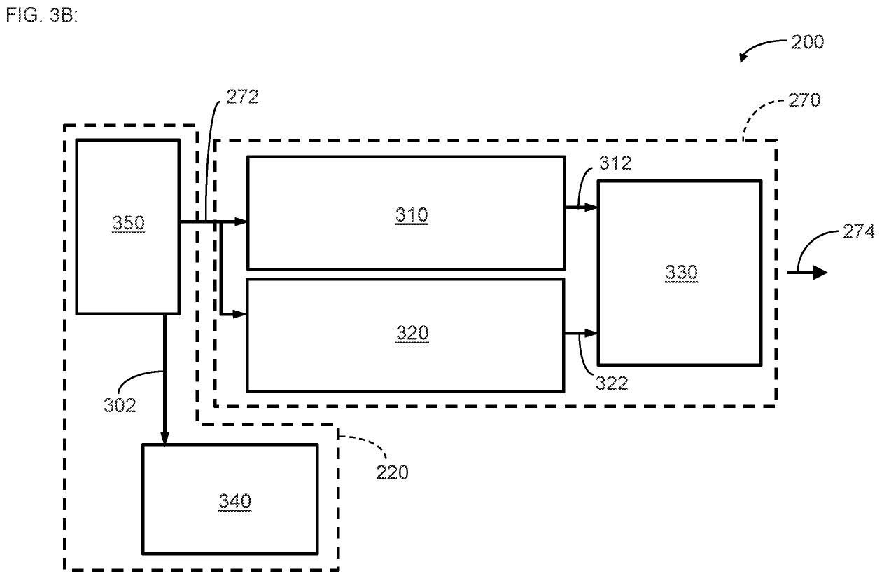

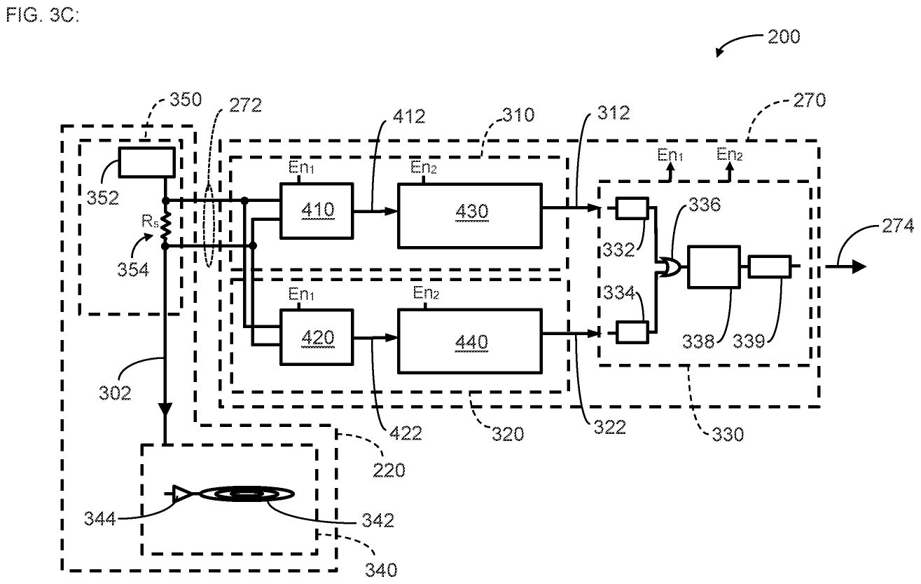

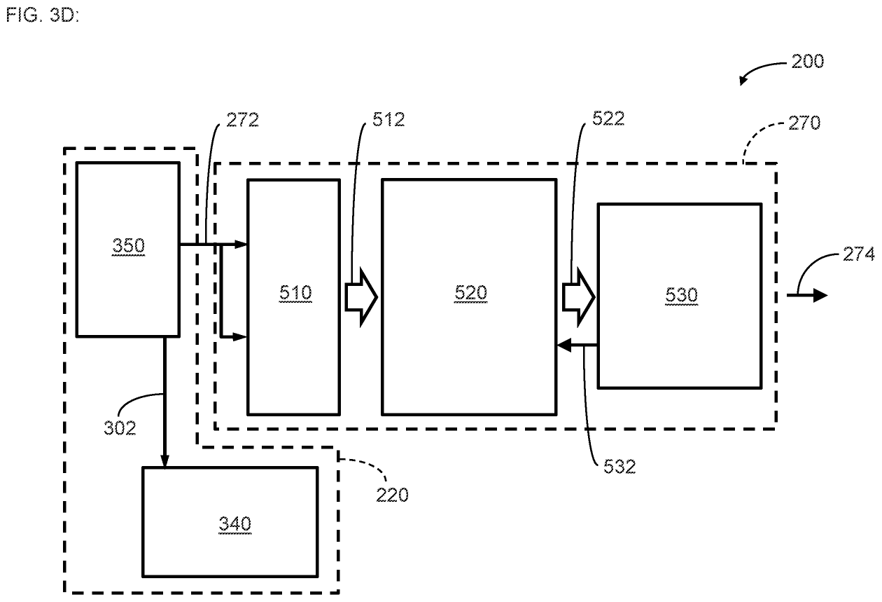

FIG. 3A schematically illustrates an example apparatus 200 comprising load modulation sensing circuitry 270 in accordance with certain implementations described herein. In certain implementations, the apparatus 200 comprises an external portion of a medical system (e.g., a portion worn by the recipient; a portion that is configured to be repeatedly attached to and detached from the recipient) and is configured to wirelessly transmit power to an internal portion of the medical system (e.g., a portion of the medical system that is implanted on or within the recipient). In certain implementations, the apparatus 200 comprises at least one magnet configured to interact with a magnetic material of the internal portion of the medical system to create an attractive magnetic force that adheres the apparatus 200 to the recipient's body in an operative position relative to the internal portion.

For example, the apparatus 200 can comprise an external portion (e.g., a sound processing unit 126; an external device 150 as in FIGS. 1A-1D) of an auditory prosthesis 100 (e.g., a cochlear implant system), the apparatus 200 configured to wirelessly provide power to an implanted stimulator unit 120. In certain implementations, the apparatus 200 is dedicated to providing power to the implanted device, while in certain other implementations, the apparatus 200 has additional functionality beyond wirelessly providing power to the implanted device (e.g., providing data and/or control signals to the implanted device to control stimulation signals provided to the recipient by the implanted device; reporting sensor information and/or status information from the implanted device to the recipient or other personnel).

In certain implementations, the load modulation sensing circuitry 270 is configured to detect variations on a DC electrical current 302 used by power transmission circuitry 220 in wireless communication with (e.g., configured to receive a data backlink from; wirelessly connected to) power receiving circuitry 240 of a device (e.g., a closely indictive coupled device). For example, the load modulation sensing circuitry 270 can be configured to detect at least positive variations on the DC electrical current 302 greater than or equal to a first threshold level, to detect at least negative variations on the DC electrical current 302 greater than or equal to a second threshold level, and to process detected positive variations and detected negative variations to generate signals indicative of load modulation of the power receiving circuitry 240 of the device (e.g., at least one internal coil 136 of an implanted device 230).

FIG. 3B schematically illustrates an example apparatus 200 in accordance with certain implementations described herein and FIG. 3C schematically illustrates additional features of the example apparatus 200 of FIG. 3B. FIG. 3D schematically illustrate another example apparatus 200 in accordance with certain implementations described herein. In the example apparatus 200 of FIGS. 3B-3C, the load modulation sensing circuitry 270 comprises first current sensing circuitry 310 configured to detect at least positive variations on the DC electrical current 302 and to generate first digital signals 312 in response thereto. The load modulation sensing circuitry 270 further comprises second current sensing circuitry 320 configured to detect at least negative variations on the DC electrical current 302 and to generate second digital signals 322 in response thereto. The load modulation sensing circuitry 270 further comprises digital processing circuitry 330 (e.g., combinatory logic circuitry) configured to receive and combine the first digital signals 312 and the second digital signals 322.

In certain implementations, the apparatus 200 is configured to detect positive changes, negative changes, and transients on the DC electrical current 302 allowing wireless communication via load modulation. For example, the first current sensing circuitry 310 can be configured to detect positive current pulses (e.g., current surges and current surge transients; pulses resulting from load modulation for small SFT values) and the second current sensing circuitry 320 can be configured to detect negative current pulses (e.g., current drops and current drop transients; pulses resulting from load modulation for medium to high SFT values), with the positive and/or negative current pulses having frequencies in a range of up to about 5 kHz. The load modulation sensing circuitry 270 can be configured to increase (e.g., maximize) the dynamic output range so as to detect pulses near saturation (e.g., towards V_{DD} or ground) and to increase (e.g., maximize) gain to improve detector sensitivity.

In certain implementations, the power transmission circuitry 220 comprises inductive coupling circuitry 340 and at least one power supply 350 configured to provide the DC electrical current 302 to the inductive coupling circuitry 340. For example, as schematically illustrated by FIG. 3C, the inductive coupling circuitry 340 can comprise at least one power transmission coil 342 (e.g., external inductive coil 130) and coil driver circuitry 344 (e.g., having one or more coil driving amplifiers; having a total amplification greater than or equal to 50×) configured to receive the DC electrical current 302 and to provide driving electrical current (e.g., DC coil driver current) to the at least one power transmission coil 342. The inductive coupling circuitry 340 can be configured to be operationally coupled by magnetic induction to at least one corresponding electrically conductive power receiving coil (e.g., antenna) of the internal portion.

As schematically illustrated by FIG. 3C, the at least one power supply 350 can comprise at least one DC voltage and/or current source 352 (e.g., providing a DC voltage in a range of 1.5 V to 4 V and a DC current in a range of less than about 50 mA) and at least one current sense resistor 354 (e.g., having a resistance Rₛ in a range of 0.1 ohm to 1 ohm; sufficiently small to substantially avoid Joule losses). The at least one current sense resistor 354 is in series between the at least one DC voltage and/or current source 352 and the inductive coupling circuitry 340 such that at least some of the DC electrical current 302 flows through the at least one current sense resistor 354 to generate a current sense voltage across the at least one current sense resistor 354, the current sense voltage indicative of the positive and negative variations on the DC electrical current 302. In certain implementations, the inductive coupling circuitry 340 and/or the at least one power supply 350 further comprises at least one capacitor configured to smooth (e.g., soften) transients on the DC electrical current 302.

In certain implementations, as schematically illustrated by FIG. 3C, the first current sensing circuitry 310 comprises at least one first amplifier 410 having a first gain and configured to receive the at least one signal 272 (e.g., current sense voltage) and to generate first voltage (e.g., analog) signals 412 having first magnitudes indicative of magnitudes of the positive variations on the DC electrical current 302. In certain implementations, as schematically illustrated by FIG. 3C, the second current sensing circuitry 320 comprises at least one second amplifier 420 having a second gain and configured to receive the at least one signal 272 (e.g., current sense voltage) and to generate second voltage (e.g., analog) signals 422 having second magnitudes indicative of magnitudes of the negative variations on the DC electrical current 302.

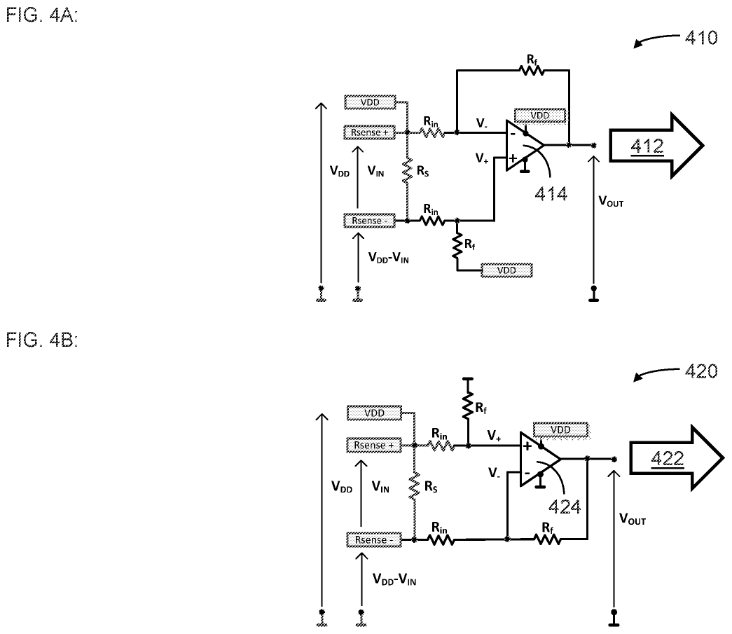

FIG. 4A schematically illustrates an example first current sensing circuitry 310 and first gain equation in accordance with certain implementations described herein. FIG. 4B schematically illustrates an example second current sensing circuitry 320 and a second gain equation in accordance with certain implementations described herein. For example, the at least one first amplifier 410 can comprise a first operational amplifier 414 and the at least one second amplifier 420 can comprise a second operational amplifier 424 (e.g., each of the operational amplifiers 414, 424 DC-coupled with the current sense resistor 354 and having a gain of at least about 50× or 36 dB) configured to amplify weak and/or short transients on the at least one signal 272 (e.g., indicative of weak and/or short transients on the DC electrical current 302). The first and second operational amplifiers 414, 424 can be configured to not go into saturation for large DC driver currents. When the DC driver current goes toward zero, the output of the first current sensing circuitry 310 goes toward VDD and the output of the second current sensing circuitry 320 goes toward ground. In certain implementations, the dynamic output range of the first current sensing circuitry 310 is configured to detect small positive pulses near ground (e.g., almost saturation) at larger DC driver currents, and the dynamic output range of the second current sensing circuitry 320 is configured to detect small negative pulses near V_{DD} (e.g., almost saturation) at larger DC driver currents.

In certain implementations, as schematically illustrated by FIG. 3C, the first current sensing circuitry 310 further comprises first comparator circuitry 430 configured to receive the first voltage signals 412 and to generate the first digital signals 312 in response to magnitudes of the first voltage signals 412 being greater than or equal to a first threshold value. In certain implementations, as schematically illustrated by FIG. 3C, the second current sensing circuitry 320 further comprises second comparator circuitry 440 configured to receive the second voltage signals 422 and to generate the second digital signals 322 in response to magnitudes of the second voltage signals 422 being greater than or equal to a second threshold value.

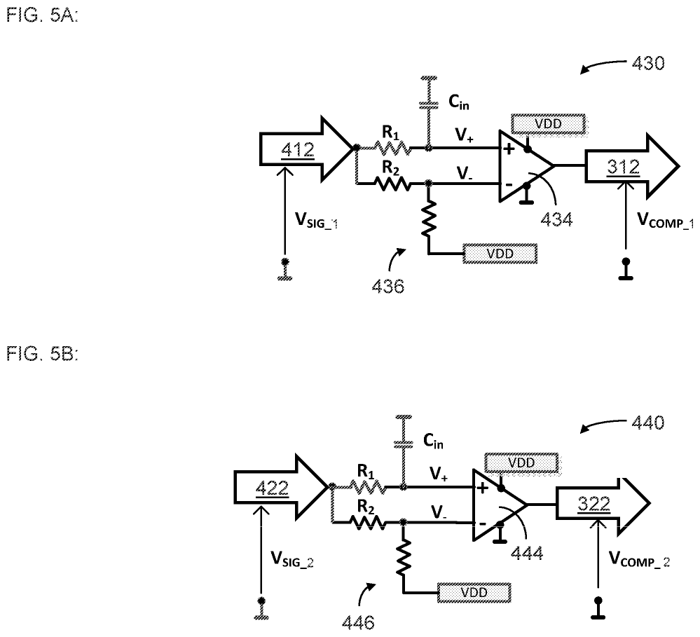

FIGS. 5A and 5B schematically illustrate an example first comparator circuitry 430 and an example second comparator circuitry 440, respectively, in accordance with certain implementations described herein. For example, the first comparator circuitry 430 can comprise a first comparator 434 (e.g., bit slicer) and the second comparator circuitry 440 can comprise a second comparator 444 (e.g., bit slicer), each of the first and second comparators 434, 444 DC-coupled with the current sense resistor 354. The first comparator circuitry 430 can be configured to compare a first scaled voltage indicative of an instantaneous magnitude of the first voltage signals 412 to the first threshold value and the second comparator circuitry 440 can be configured to compare a second scaled voltage indicative of an instantaneous magnitude of the second voltage signals 422 to the second threshold value. The first threshold value can be substantially equal to a first average DC voltage output of the at least one first amplifier 414 (e.g., from a passive integrator or low pass circuit) and the second threshold value can be substantially equal to a second average DC voltage output of the at least one second amplifier 424 (e.g., from a passive integrator or low pass circuit). The instantaneous magnitudes of the first voltage signals 412 and the second voltage signals 422 can each be scaled by corresponding squelch circuits 436, 446 each comprising at least one variable resistor. For example, the squelch circuits 436, 446 can be programmed such that the first comparator circuitry 430 and the second comparator circuitry 440 detect current variations with a sensitivity of at least 50 mV (e.g., in a range of 1 mA to at least 10 mA through a current sense resistor 354 of 1 ohm, with step sizes of 0.1 mA±100%).

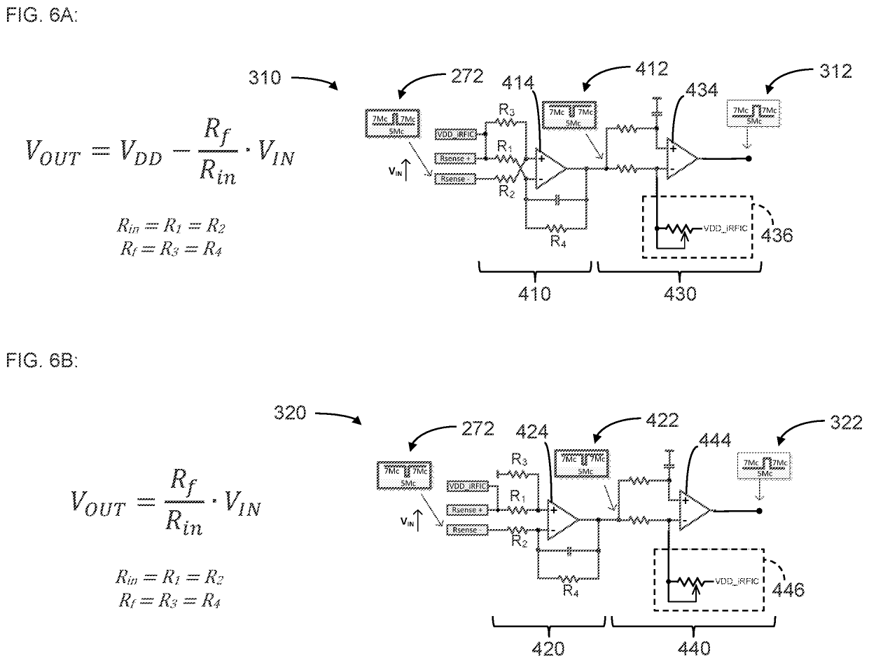

FIG. 6A schematically illustrates an example first current sensing circuitry 310 comprising the at least one first amplifier 410 of FIG. 4A and the first comparator circuitry 430 of FIG. 5A in accordance with certain implementations described herein. FIG. 6B schematically illustrates the second current sensing circuit 320 comprising the at least one second amplifier 420 of FIG. 4B and the second comparator circuitry 440 of FIG. 5B in accordance with certain implementations described herein. In certain other implementations, either the first current sensing circuitry 310 and/or the second current sensing circuitry 320 further comprises an analog-to-digital converter configured to receive the first voltage signals 412 and/or the second voltage signals 422, respectively, and to provide a measurement of the DC electrical current 302 (e.g., to monitor an absolute level of the DC coil drivers).

For example, as shown in FIG. 6A, the at least one first amplifier 410 can be configured to respond to positive pulses of the at least one signal 272 (e.g., corresponding to toggles of the frequency detunings of the power receiving circuitry 240 with a small SFT value) by generating negative pulses of the first voltage signals 412 (V_{OUT}) which the first comparator circuitry 430 respond to by generating positive digital pulses of the first digital signals 312. For another example, as shown in FIG. 6B, the at least one second amplifier 420 can be configured to respond to negative pulses of the at least one signal 272 (e.g., corresponding to toggles of the frequency detunings of the power receiving circuitry 240 with a medium or large SFT value) by generating negative pulses of the second voltage signals 422 (V_{OUT}) which the second comparator circuitry 440 respond to by generating positive digital pulses of the second digital signals 322.

As schematically illustrated by FIG. 3C, the digital processing circuitry 330 comprises first level translator circuitry 332 configured to receive the first digital signals 312, second level translator circuitry 334 configured to receive the second digital signals 322, “OR” logic circuitry 336 configured to receive the outputs of the first and second level translator circuitry 332, 334, monostable circuit 338 configured to receive the output of the “OR” logic circuitry 336, and digital logic circuitry 339 configured to generate a data_valid signal. The first and second level translator circuitry 332, 334 (e.g., level shifters) can be configured to translate the first digital signals 312 and/or the second digital signals 322 from a logic level or voltage domain of the first and second current sense circuitry 310, 320 to a logic level or voltage domain of the digital processing circuitry 330. The “OR” logic circuitry 336 is configured to combine (e.g., digitally add) the first and second digital signals 312, 322 to generate a series of digital pulses (e.g., pulse train). The monostable circuit 338 is configured to revise (e.g., stabilize) the series of digital pulses by replacing multiple pulses within a predetermined time window width (e.g., in a range of 50 microseconds to 700 microseconds) with a single digital pulse. In this way, the monostable circuit 338 can filter out multiple pulses that can occur during a single load modulation event (e.g., a single toggle of frequency detuning or resistive load from a first value to a second value and back to the first value), leaving only a single pulse corresponding to the single load modulation event.

In certain implementations, the digital processing circuitry 330 is configured to provide enable signals to the first and second current sense circuitry 310, 320. For example, as shown in FIG. 3C, the digital processing circuitry 330 can be configured to provide first enable signals En₁ to the at least one first amplifier 410 and to the at least one second amplifier 420 and can be configured to provide second enable signals En₂ to the first and second comparator circuitry 430, 440. The first and second enable signals can be used to provide a disable functionality to save power.

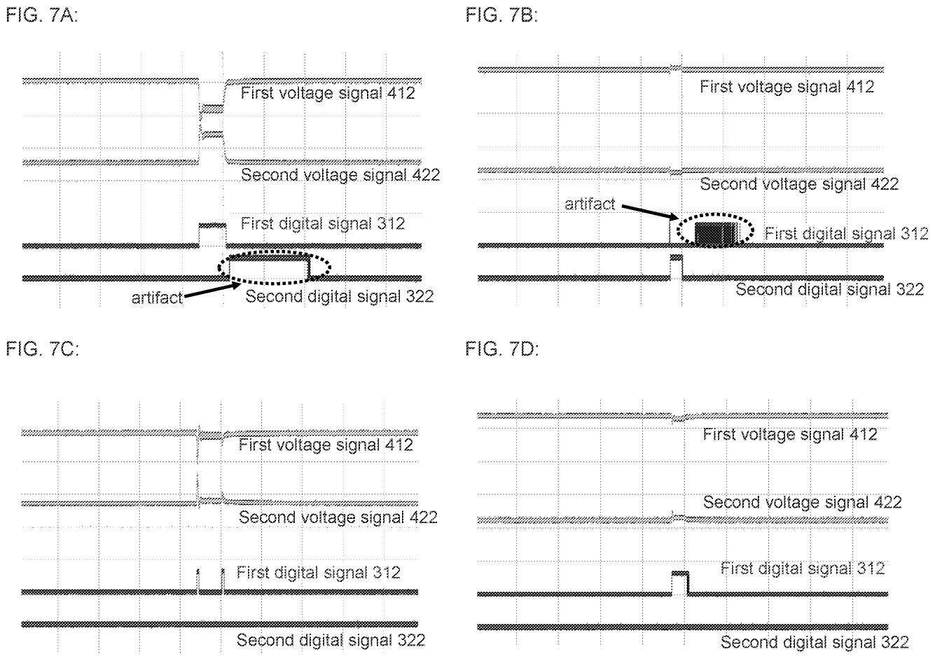

FIGS. 7A-7D schematically illustrate plots of the first voltage signals 412, second voltage signals 422, first digital signals 312 and second digital signals 322 from configurations configured to model various SFT values in accordance with certain implementations described herein. FIG. 7A shows the signals corresponding to a single load modulation event for a first SFT value for which the single load modulation event corresponds to a large increase (e.g., large surge) of the DC driver current 302. FIG. 7B shows the signals corresponding to a single load modulation event for a second SFT value for which the single load modulation event corresponds to a decrease (e.g., drop) of the DC driver current 302. FIG. 7A shows an artifact in the second digital signal 322 and FIG. 7B shows an artifact in the first digital signal 312. These artifacts are due to the recharge effect of an RC integrator next to an incoming load modulation pulse. Using a monostable circuit 338 having a predetermined time window width of about 500 microseconds can reduce (e.g., prevent; avoid) deleterious effects from such artifacts. FIG. 7C shows the signals corresponding to a single load modulation event for a third SFT value for which the single load modulation event corresponds to a surge transient of the DC driver current 302 and FIG. 7D shows the signals corresponding to a single load modulation event for a fourth SFT value for which the single load modulation event corresponds to a small increase (e.g., small surge) of the DC driver current 302.

FIG. 3D schematically illustrate another example apparatus 200 in accordance with certain implementations described herein. The load modulation sensing circuitry 270 of FIG. 3D comprises at least one amplifier 510 configured to receive the at least one signal 272 (e.g., current sense voltage) and to generate analog voltage signals 512 having magnitudes indicative of magnitudes of the positive and negative variations on the DC electrical current 302. The load modulation sensing circuitry 270 of FIG. 3D further comprises analog-to-digital converter (ADC) circuitry 520 configured to convert the voltage signals 512 to digital signals 522. The load modulation sensing circuitry 270 of FIG. 3D further comprises digital processing circuitry 530 (e.g., logic circuitry) configured to, in response to the digital signals 522, detect the positive and negative variations and to generate, in response thereto, the digital signals 274 indicative of the load modulation. For example, the digital processing circuitry 530 can comprise a central processing unit (CPU) or digital signal processor (DSP) comprising logic circuitry and/or processing circuitry, program memory circuitry, data memory, and clock circuitry. In certain implementations, the clock circuitry generates a clock signal 532, the ADC circuitry 520 is configured to respond to the clock signal 532 by sampling the voltage signals 512 at a sampling rate (e.g., 100 Hz to 100 kHz).

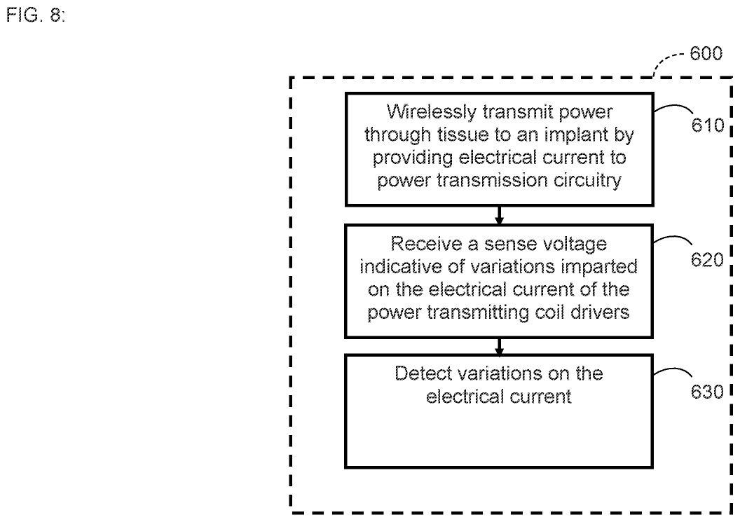

FIG. 8 is a flow diagram of an example method 600 in accordance with certain implementations described herein. While the method 600 is described by referring to some of the structures of the example apparatus 200 of FIGS. 2A-2B, 3A-3D, 4A, 4B, 5A, 5B, 6A, and 6B, other apparatus and systems with other configurations of components can also be used to perform the method 600 in accordance with certain implementations described herein.

In an operational block 610, the method 600 comprises wirelessly transmitting power 222 through tissue (e.g., a portion of a recipient's body 210) to an implant (e.g., implanted device 230) on or within a recipient's body by providing electrical current 302 to power transmission circuitry 220 inductively coupled to power reception circuitry 240 of the implant. For example, the power can be transmitted via a magnetic induction link that transfers electric power from an external portion of a medical device or system (e.g., an auditory or visual prosthesis system; cardiac pacemaker or defibrillator system) with the electric power received by an internal portion (e.g., implanted component) of the medical device or system.

In an operational block 620, the method 600 further comprises receiving a sense voltage (e.g., at least one signal 272) indicative of variations imparted onto the electrical current 302 provided to the power transmission circuitry 220 (e.g., electrical current of the power transmitting coil drivers) by controlled adjustments of a resonant frequency and/or a resistive load of the power receiving circuitry 240.

In an operational block 630, the method 600 further comprises detecting variations on the electrical current 302. In certain implementations, said detecting comprises generating a plurality of backlink data pulses. For example, said generating the plurality of backlink data pulses can comprise generating a plurality of first digital pulses 312 indicative of positive current variations on the DC electrical current 302 and/or generating a plurality of second digital pulses 322 indicative of negative current variations on the DC electrical current 302, and applying digital logic to the first plurality of digital pulses 312 and/or the second plurality of digital pulses 322. Said generating the plurality of first digital pulses 312 can comprise amplifying positive analog pulses on the sense voltage, comparing a positive magnitude of each amplified positive analog pulse to a positive threshold value, and generating a first digital pulse 312 of the plurality of first digital pulses 312 in response to the amplified positive analog pulse having a positive voltage magnitude greater than the positive threshold value. Said generating the plurality of second digital pulses 322 can comprise amplifying negative analog pulses on the sense voltage, comparing a negative magnitude of each amplified negative analog pulse to a negative threshold value, and generating a second digital pulse 322 of the plurality of second digital pulses 322 in response to the amplified negative analog pulse having a negative voltage magnitude greater than the negative threshold value. For another example, said generating the plurality of backlink data pulses can comprise amplifying analog pulses indicative of positive and/or negative current variations on the DC electrical current 302 (e.g., positive and/or negative analog pulses on the sense voltage), converting the amplified analog pulses to digital pulses, comparing the digital pulses to at least one threshold value, and applying digital processing logic to the digital pulses.

Said generating the plurality of backline data pulses can further comprise generating a filtered pulse train (e.g., digital signals 274) by detecting two or more pulses of the digital pulses that are separated from one another by a time period less than or equal to a threshold time period and replacing the two or more pulses by a single pulse. In certain implementations, the method 600 further comprises decoding the filtered pulse train to extract information received from the implant.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having an external portion of the implantable medical device wirelessly receive information from an implanted portion of the implantable medical device while the external portion wirelessly transmits power to the implanted portion.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
load modulation sensing circuitry configured to detect variations on a DC electrical current used by power transmission circuitry configured to be in wireless communication with power receiving circuitry of a device, the load modulation sensing circuitry configured to detect at least positive variations on the DC electrical current greater than or equal to a first threshold level, to detect at least negative variations on the DC electrical current greater than or equal to a second threshold level, and to process detected positive variations and detected negative variations to generate signals indicative of load modulation of the power receiving circuitry of the device.

2. The apparatus of claim 1, wherein the power transmission circuitry comprises inductive coupling circuitry and at least one power supply configured to provide the DC electrical current to the inductive coupling circuitry, the inductive coupling circuitry comprising at least one power transmission coil and coil driver circuitry configured to receive the DC electrical current and to provide driving electrical current to the at least one power transmission coil.

3. The apparatus of claim 2, wherein the at least one power supply comprises at least one DC voltage and/or current source and at least one current sense resistor in series between the at least one DC voltage and/or current source and the inductive coupling circuitry such that at least some of the DC electrical current flows through the at least one current sense resistor to generate a current sense voltage across the at least one current sense resistor, the current sense voltage indicative of the positive and negative variations.

4. The apparatus of claim 3, wherein the load modulation sensing circuitry comprises:
first current sensing circuitry configured to detect the at least positive variations and to generate first digital signals in response thereto;
second current sensing circuitry configured to detect the at least negative variations and to generate second digital signals in response thereto; and
combinatory logic circuitry configured to receive and combine the first digital signals and the second digital signals to generate the signals indicative of the load modulation.

5. The apparatus of claim 4, wherein the first current sensing circuitry comprises:
at least one first amplifier configured to receive the current sense voltage and to generate first voltage signals having first magnitudes indicative of magnitudes of the positive variations on the DC electrical current; and
first comparator circuitry configured to receive the first voltage signals and to generate the first digital signals in response to magnitudes of the first voltage signals being greater than or equal to a first threshold value;
and the second current sensing circuitry comprises:
at least one second amplifier configured to receive the current sense voltage and to generate second voltage signals having second magnitudes indicative of magnitudes of the negative variations on the DC electrical current; and
second comparator circuitry configured to receive the second voltage signals and to generate the second digital signals in response to magnitudes of the second voltage signals being greater than or equal to a second threshold value, wherein the first threshold value is substantially equal to a first average DC voltage output of the at least one first amplifier and the second threshold value is substantially equal to a second average DC voltage output of the at least one second amplifier.

6. The apparatus of claim 5, wherein the first comparator circuitry is configured to compare a first scaled voltage indicative of an instantaneous magnitude of the first voltage signals to the first average DC voltage output of the at least one first amplifier and the second comparator circuitry is configured to compare a second scaled voltage indicative of an instantaneous magnitude of the second voltage signals to the second average DC voltage output of the at least one second amplifier.

7. The apparatus of claim 1, wherein the combined first and second digital signals are indicative of backlink data received by the apparatus from the power receiving circuitry.

8. The apparatus of claim 3, wherein the load modulation sensing circuitry comprises:
at least one amplifier configured to receive the current sense voltage and to generate voltage signals having magnitudes indicative of magnitudes of the positive and negative variations on the DC electrical current;
analog-to-digital converter (ADC) circuitry configured to detect the voltage signals; and
digital processing circuitry configured to detect the positive and negative variations and to generate, in response thereto, the signals indicative of the load modulation.

9. The apparatus of claim 8, wherein the ADC circuitry is configured to sample the voltage signals at a sampling rate.

10. The apparatus of claim 1, further comprising controller circuitry configured to receive the signals indicative of the load modulation, to extract information therefrom, and to use the information.

11. The apparatus of claim 1, wherein the apparatus further comprises the power transmission circuitry and at least one power supply configured to provide the DC electrical current to the power transmission circuitry.

12. The apparatus of claim 1, wherein the apparatus comprises an external portion of a medical device, and the power receiving circuitry is within an implantable portion of the medical device configured to be implanted on or within a recipient's body with tissue between the external portion and the implantable portion.

13. The apparatus of claim 12, wherein the medical device comprises an acoustic prosthesis.

14. A method comprising:
wirelessly transmitting power through tissue to an implant on or within a recipient's body by providing electrical current to power transmission circuitry inductively coupled to power reception circuitry of the implant;
receiving a voltage indicative of variations imparted onto the electrical current by controlled adjustments of a resonant frequency and/or a resistive load of the power receiving circuitry; and
detecting the variations on the electrical current, said detecting comprising:
in response to the voltage, generating signals indicative of negative current variations and/or positive current variations on the electrical current; and
deriving data from the signals.

15. The method of claim 14, wherein said generating the signals comprises using analog-to-digital converter circuitry configured to sample the voltage and said deriving the data comprises using digital processing circuitry.

16. The method of claim 14, wherein said generating the signals comprises generating a plurality of first digital pulses indicative of the negative current variations and/or generating a plurality of second digital pulses indicative of the positive current variations, and said deriving the data comprises generating a pulse train by combining the first digital pulses and the second digital pulses.

17. The method of claim 16, further comprising:
generating a filtered pulse train by detecting two or more pulses of the combined pulse train that are separated from one another by a time period less than or equal to a threshold time period and replacing the two or more pulses by a single pulse; and
decoding the filtered pulse train to extract information received from the implant.

18. The method of claim 16, wherein said generating the plurality of first digital pulses comprises amplifying negative analog pulses on the voltage, comparing a negative magnitude of each amplified negative analog pulse to a negative threshold value, and generating a first digital pulse of the plurality of first digital pulses in response to the amplified negative analog pulse having a negative voltage magnitude greater than the negative threshold value and/or said generating the plurality of second digital pulses comprises amplifying positive analog pulses on the voltage, comparing a positive magnitude of each amplified positive analog pulse to a positive threshold value, and generating a second digital pulse of the plurality of second digital pulses in response to the amplified positive analog pulse having a positive voltage magnitude greater than the positive threshold value.

19. An apparatus comprising:
at least one coil driver comprising at least one current sense resistor;
at least one power transmitting coil configured to receive an electrical current from the at least one sense resistor, the at least one power transmitting coil configured to be in inductive communication with a device; and
load modulation sensing circuitry configured to detect variations of a voltage across the at least one current sense resistor.

20. The apparatus of claim 19, wherein the load modulation sensing circuitry comprises:
at least one amplifier configured to receive the voltage and to generate voltage signals having magnitudes indicative of magnitudes of positive and negative variations of the voltage;
analog-to-digital converter (ADC) circuitry configured to detect the voltage signals; and
digital processing circuitry configured to detect the positive and negative variations and to generate, in response thereto, signals indicative of the load modulation.

21. The apparatus of claim 19, wherein the load modulation sensing circuitry comprises:
first circuitry configured to detect at least negative variations of the voltage and to generate first digital signals in response thereto;
second circuitry configured to detect at least positive variations of the voltage and to generate second digital signals in response thereto; and
combinatory logic circuitry configured to receive and respond to the first digital signals and the second digital signals by generating a digital pulse train.

22. The apparatus of claim 21, wherein the first circuitry is configured to generate first analog signals having first magnitudes indicative of magnitudes of positive variations of the voltage and to generate the first digital signals in response to the magnitudes of the positive variations of the voltage being greater than or equal to a first threshold value, and the second circuitry is configured to generate second analog signals having second magnitudes indicative of magnitudes of negative variations of the voltage and to generate the second digital signals in response to the magnitudes of the negative variations of the voltage being greater than or equal to a second threshold value.
